(19) United States
(12) Patent Application Publication
(10) Pub. No.: US 2003/0224074 A1
(43) Pub. Date: Dec. 4, 2003

(54) Composition for the protection and regeneration of nerve cells containing the extract of Scutellaria Radix

(75) Inventors: Byung-Kil Choe, Seo-gu (KR); Yun-Hee Kim, Seoul (KR); Soo-Kyung Kim, Jung-gu (KR); Jung-Su Lim, Seoul (KR); Hyo-Sup Kim, Namdong-gu (KR); Dae-Sung Park, Seoul (KR); Chi-Young Chang, Bucheon-si (KR)

Correspondence Address: HESLIN ROTHENBERG FARLEY & MESITI PC, 5 COLUMBIA CIRCLE, ALBANY, NY, 12203, US

(73) Assignee: EUGENBIO INC., Asan-si (KR)

(21) Appl. No.: 10/389,677

(22) Filed: Mar. 14, 2003

Related U.S. Application Data
(63) Continuation of application No. PCT/KR02/01315, filed on Jul. 11, 2002.

(30) Foreign Application Priority Data
Jul. 11, 2001 (KR) 2001-0041688
Jul. 11, 2002 (KR) 2002-0040184

(51) Int. Cl. A61K 35/78

(52) U.S. Cl. 424/741

(57) ABSTRACT

Disclosed is a composition for protecting nerve cells, promoting nerve cell growth and regenerating nerve cells comprising a Scutellaria Radix extract. The composition has excellent protective effects against apoptosis of neuronal stem cells and differentiated nerve cells, a positive effect of inducing the regeneration of nerve cells, a regenerative effect on neurites, a neuroregenerative effect on brain nerves and peripheral nerves, a reformation effect on neuromuscular junctions, and a protective effect agaisnt apoptosis of nerve cells and a neuroregenerative effect in animals suffering from dementia and brain ischemia. Therefore, the composition can be used as a therapeutic agent for the prevention and treatment of nerodegenerative diseases, ischemic nervous diseases or nerve injuries, and for the improvement of learning capability.

### BACKGROUND OF THE INVENTION

1. Field of the Invention

The present invention relates to a composition for protecting nerve cells, promoting nerve cell growth and regenerating nerve cells comprising a Scutellaria Radix extract. Further, the present invention relates to a composition for drugs and functional foods useful in the prevention and treatment of nervous diseases or nerve injuries comprising a Scutellaria Radix extract.

The composition according to the present invention can be used as therapeutic agents for the prevention and treatment of neurodegenerative diseases, ischemic nervous diseases or brain injuries, and for the improvement of learning capability.

2. Description of the Related Art

Synapses are the connection points between nerve cells, and one nerve cell connects to 1000˜5000 other nerve cells on average. It is estimated that since at least 10¹¹ nerve cells exist in the human brain, there are at least 10¹⁴ synapses in the human brain. All complex and various brain functions, for example thoughts, sensations, memory, learning and actions, cannot be understood without consideration of these neural networks. Synaptic connections are essential to nerve cell survival. Special functions according to the connections between nerve cells make it possible to express high-level brain functions intrinsic to humans. In particular, it is known that once the central nervous system is damaged, its regeneration is very difficult. Many ideas and attempts for treating damaged nerve tissues or chronic degenerative diseases have been made in various ways. In the 1940's, Hamburger and Levi-Montalcini discovered an unidentified substance indispensable for survival of motor neurons in the differentiation process of Chick embryo limb, and proposed the neurotrophic factor hypothesis. Based on the hypothesis, NGF (nerve growth factor) was first discovered, and discoveries of neurotrophic factors such as BDNF (brain-derived neurotrophic factor), NT-3 (neurotrophic factor-3), etc., followed. Further, it was found in some transgenic animal experiments which types of nerve growth factors are necessary for survival of each differentiated nerve cell population. Also, it was found that not only neurotrophins but also some cytokines are involved in nerve cell survival. When neurotrophins or cytokines are not supplied or receptors for these neurotrophins or cytokines are not expressed in the cells, nerve cells die. There are two nerve cell death pathways, like all other cells: necrosis and apoptosis. Necrosis and apoptosis have different morphological and molecular biological characteristics. When an axon is cut (axotomy), a part attached to the cell body and a terminal forming a synapse are separated each other. Such axotomy leads to not only synaptic denaturation due to cut off of supply of protein factors from target cell body, but also synaptic detachment. That is, regeneration is a key to nerve cell survival. Dead nerve cells are replaced with glial cells in the peripheral nervous system, and astrocytes or microglias in the central nervous system, in a process called “synaptic stripping”. In addition, immune system cells such as monocytes, macrophages, etc., can replace the dead nerve cells, depending on the extent of damages. Many theories explaining mechanisms of physical injuries to nerve cells, acute neurotoxicity, acute and chronic nervous disorders, dementia, epilepsy., etc. have been introduced, but these theories all have a common point. That is, these diseases affect nerve cells and supporting tissue cells thereof. These cells extend horizontally and perpendicularly to form many dendrites and axons, which form many neural networks. Abnormalities in the neural nets lead to deregulation in signal transmission and cause various cranial nervous system diseases. The glutamatergic neural net responding to glutamate, an excitatory neurotransmitter, is a neural net to which has drawn attention in terms of development of acute and chronic cranial nervous diseases.

All mammalian brains develop a systematic neural network through a series of division, differentiation, survival and apoptosis of neuronal stem cells, and synaptic formation, thereby performing complex brain functions. In the adult brain, cranial nerve cells produce many substances necessary for nerve growth to make their axons and dendrites grow. Therefore, as new learning and memories are introduced, synaptic connections and neural networks are continuously remodeled. In the differentiation and synaptic formation of nerve cells, cells not receiving target-derived survival factors such as nerve growth factors die, and cell death due to stress and cytotoxic agents is a major cause of degenerative brain diseases. When the peripheral nervous system is injured, the differentiation of axons requires a long time, unlike the central nervous system. Rear axons of the injured nerve cells undergo Wallerian degeneration, the cell bodies undergo axonal regrowth, and Schwann cells are regenerated through a series of divisions to determine target nerves by survival and apoptosis, and redifferentiation, etc.

It was recently shown that neuronal stem cells exist in the adult brain. The development and differentiation of the stem cells in the adult brain lead to the regeneration of nerve cells (Johansson, C. B., Momma S., Clarke D. L., Risling M., Lendahl U., and Frisen J. (1999) Identification of a neural stem cell in the adult mammalian central nervous system, Cell 96, 25-34). Neuronal stem cells are mainly found in the subventricular zones of striatum adjacent to lateral ventricles. Neural stem cells in the subgranular zones at dentate gyrus of the hippocampus divide to form granule cells (van Praag et al., Nature 415, 1031-1034 (2002)). Therefore, increased development and differentiation of neuronal stem cells can promote nerve regeneration.

During the developmental stage of the mammalian brain, more than half of developed nerve cells die. In addition, such nerve cell death takes place not only in the nervous system diseases, in particular of aged nervous systems, but also in the normal adult brain (Yuan and Yankner, Nature. 407, 802-809 (2000)). Therefore, apoptosis of nerve cells is a major problem in all nervous system diseases including degenerative brain diseases in the central nervous system and spinal cord and peripheral nervous system injuries. In Europe, transplantation of fetal neuronal stem cells into patients with degenerative brain disease, in particular, Parkinson's disease, has been clinically tried. After transplantation, patients exhibited significant improvement. However, 3 months after transplantation, since most of transplanted cells die, there is a need to continuously transplant neuronal stem cells into patients (Olanow C. W., Kordower J. H., Freeman T. B. (1996) Fetal nigral transplantation as a therapy for Parkinson's disease. Trends Neurosci. 19, 102-109.) In order to survive in the nervous system, transplanted cells must differentiate into their compatible nerve cells to form synapses together with target cells, and participate in electrical signal transmission to continuously receive survival factors from the target cells.

Although many studies on nerve cell apoptosis have been undertaken in differentiated nerve cells, little is known about substances to hinder nerve cell apoptosis, in particular in neuronal stem cells.

Neuronal stem cells divide into other stem cells or cells to be differentiated. At this time, cells suffering from false cell division and unnecessary cells experience cell death. Surviving cells are classified according to types of cells they are differentiated into. Neuronal precursors or neuroblasts, which are differentiated into nerve cells, are differentiated into cells secreting suitable neurotransmitters. Glial precursors, which are differentiated into glial cells, are differentiated into astrocytes and oligodendrocytes. These are cells assisting nerve cells. Astrocytes mechanically and metabolically support nerve cells, and comprise 70˜80% of adult brain cells. Oligodendrocytes insulate axons and produce myelin to increase the rate of transmission of signals. Neuronal stem cells in the central nervous systems of fetus and adult can be differentiated into three types of brain cells, depending on environment of brain tissues and type of signals transmitted to neuronal stem cells.

It was reported that there are three types of cells as stem cells in the central nervous system. These cells all exist in the adult rodent brain, and it is believed that the cells exist in the adult human brain. One area containing these cells exists in the brain tissues adjacent to ventricles known as ventricular zones and subventricular zones. Ventricle is spaces through which cerebrospinal fluid can flow. During fetal neurogenesis, rapid cell division takes place in the tissues around the ventricles. In the adult, stem cells around ventricles can exist, but the tissues are very small. The second area in which stem cells exist is not found in humans. The area is rostral migratory stream connecting lateral ventricles and olfactory bulbs in rodents. The third area is the hippocampus, which is associated with memory formation, and exists in both the adult rodent and human brains.

Stem cells in the hippocampus exist in the subgranular zones of dentate gyrus. When labeling dividing cells with BrdU (bromodeoxyuridine) in rats, about half of the labeled cells are differentiated into granule cells of dentate gyrus, and 15% are differentiated into glial cells, and the rest do not have particular phenotypes.

Some BrdU-labeled cells in dentate gyrus of human and rat express nerve cell markers such as NeuN, neuron-specific enolase, calbindin., etc. These nerve-like cells are similar to granule cells of dentate gyrus in terms of morphology. The other BrdU-labeled cells express GFAP, which is an astrocyte marker. Recent study has revealed that as a result of analyzing BrdU-labeled cells in the brain tissues of five cancer patients (age 57˜72 years) for the purpose of diagnosing, BrdU-labeled cells were most commonly found in the brain of the oldest patient. From this finding, it can be seen that the formation of nerve cells in the hippocampus continues until death.

It is known that nerve growth factors are involved in division, differentiation and apoptosis of neuronal stem cells, differentiation of neuronal stem cells into nerve cells and glial cells, and synaptic formation in the development of mammalian nerves.

The receptors for the nerve growth factors are tyrosine kinases. Fibroblast growth factors (FGFs) were first found to be growth factors promoting the division of neuroectoderm and mesoderm-derived cells. FGFs are classified into acidic FGFs (aFGF) and basic FGFs (bFGF) in terms of their isoelectric points. Membrane-associated proteoglycans bind to low-affinity binding sites of FGF receptors, and are essential to FGF's binding with a high-affinity binding site. It is known that almost all high-affinity receptors are receptor-tyrosine kinases, and FGF is bound thereto to form a dimer which causes tyrosine autophosphorylation and transmits signals in 3T3 fibroblast and platelet. FGF receptors express 4 genes into various transcripts by alternative splicing. The receptors can bind with at least one FGF family member, and their ligand binding specificities are determined by their types and splicing forms. FGFs have mitogen activity and induce cell differentiation. The treatment of pheochromocytomas (PC12) with FGF causes their differentiation into cells having neuronal phenotype.

Little is known about the signal transmission system of FGF receptors. When the primary cells of the hippocampus and PC12 cells are treated with FGF receptors, tyrosine phosphorylation increases and p42 MAP kinase (ERK2) and p44 MAP kinase (ERK1), which are mitogen-activated protein kinases (MAP kinase), are activated. Further, it is known that bFGF induces transcription factor such as c-fos. It has been found that FGF increases the survival of the hippocampus and cerebral cortex nerves, and neurite outgrowth in primary nerve cell culture of white rat brain, and decreases excitotoxicity by glutamate. mRNAs of FGF receptors are mainly found in the adult rat brain, in particular in primary cultured nerve cells of developing rat brain and hippocampus. Furthermore, it is known that FGF increases the survival of retinal optic nerves during the development of Xenopus retinal optic nerve cells, and in particular the expression of FGF is drastically increased in a short period of time.

Primary culture of nervous stem cells in E16, in which hippocampal pyramidal nerve cells develop, and treatment of the primary culture with FGF, a nerve growth factor, increase cell division. At this time, 30% of stem cells differentiate into nerve cells, and the remaining stem cells differentiate into glial cells. McKay's group reported that the treatment of with PDGF mainly leads to the differentiation into nerve cells (80%) and the differentiated nerve cells express neuronal markers. They also reported that treatment with FGF and EGF, followed by treatment with CNTF, leads to differentiation into astrocytes, and treatment with thyroid hormone T3 promotes differentiation into oligodendrocytes. These findings mean that PDGF acts as a neurotrophic factor in the early stage of primitive nerve cell development to determine the fate of neuronal cells.

The present inventors found that the treatment of the hippocampal primitive nerve cell line (HiB5) with PDGF and FGF inhibits apoptosis of cells and influences the differentiation into nerve cells or glial cells (Kwon, Y. Kim (1997) Expression of brain-derived neutrophic factor mRNA stimulated by basic fibroblast grwoth factor and platelet-derived growth factor in rat hippocampal cell line, Mol. Cells 7, 320-325.).

Nerve growth factors initiate the division of nerve stem cells, regulate the number of divided cells into apoptosis, initiate the differentiation of divided cells, induce the survival of cells orthodromically moving toward target-derived growth factors and the apoptosis of cells moving in a false direction to regulate the survival of presynaptic nerve cells, and regulate synaptic formation and synaptic remodeling. Since the human central nervous system and peripheral nervous system are hard to regenerate, patients with degenerative brain diseases, and persons crippled due to industrial accidents, traffic accidents and wars, have been social problems. Therefore, special attention has been paid to studies on the regeneration of nervous systems.

Scutellaria Radix is a perennial plant belonging to the class dicotyledoneae, order tubiflorales, family Labiatae. The root of Scutellaria Radix has been traditionally used as an antipyretic, a diuretic, an antidiarrhotica, a cholagogue and an antiphlogistic in Oriental medicine, and Scutellaria Radix stew has been used to treat diarrhea, anorexia and colic due to acute gastroenteritis.

Korean Laid-open Patent No. 2001-0081188 (US 2001/0026813 A1) discloses the protective activity of a Scutellaria Radix extract against the damage to neuronal cells and its therapeutic mechanism in PC12 cell line using an ischemic model.

The present inventors identified the effects of a Scutellaria Radix extract on differentiation and regeneration of nerve cells, in addition to the protective activity of a Scutellaria Radix extract against the damage to brain nerve cells. Further, they first identified protective, regenerative, differentiative and reformation effects of a Scutellaria Radix extract on neuronal stem cells and peripheral nerve cells.

### SUMMARY OF THE INVENTION

Therefore, it is an object of the present invention to provide a drug and food composition for protecting nerve cells, promoting the differentiation of nerve cells including neuronal stem cells and regenerating nerve cells, comprising a Scutellaria Radix extract.

It is another object of the present invention to provide a drug and food composition for preventing and treating nervous diseases or nerve injuries, comprising a Scutellaria Radix extract.

It is yet another object of the present invention to provide a drug and food composition for preventing and treating neurodegenerative diseases, ischemic nervous diseases and central or peripheral nerve injuries due to accidents, and for improving learning capability, comprising a Scutellaria Radix extract.

The composition according to the present invention is useful for preventing and treating physical injuries to nervous systems, degenerative and ischemic cranial nerve injuries, and peripheral nerve injuries.

The present inventors identified the effects of the Scutellaria Radix extract on differentiation and regeneration of nerve cell lines including neuronal stem cells cultured in vitro. In in vivo experiments, the present inventors identified the inhibitory effect of the Scutellaria Radix extract against apoptosis of cells, and the protective effect on nerve cells in apoptosis-induced animal models, by treating with a neurotoxin. Further, they identified the effect of the Scutellaria Radix extract on regeneration of injured peripheral nerves in peripheral nerve-injured animal models. Specifically, the present inventors examined the effects of the Scutellaria Radix extract on differentiation and regeneration of nerve cells in vitro, by treating human neuroblastoma (SH-SY5Y), white rat hippocampus-derived neuronal stem cells (HiB5), and rat-derived PC12 cell cultures with the Scutellaria Radix extract. In order to investigate the apoptosis of nerve cells as a cause of all neurodegenerative diseases, after treating an experimental animal with MK-801 to induce apoptosis of brain cells, inhibitory effect of the Scutellaria Radix extract against apoptosis of cells and the protective effect against the stress were identified. In addition, using sciatic nerve-crushed animal models, the effect of the Scutellaria Radix extract on regeneration of peripheral nerves was examined.

From these experiments, it was confirmed that the Scutellaria Radix extract has excellent differentiative and regenerative effects on nerve cells including neuronal stem cells, an inhibitory effect against apoptosis of nerve cells in vivo, and a regenerative effect on injured peripheral nerves.

Therefore, it is expected that the Scutellaria Radix extract will be useful for preventing and treating nervous system disorders, degenerative brain diseases including dementia, nervous system diseases, and central nerve injuries and peripheral nerve injuries by traffic accidents, etc.

### DESCRIPTION OF THE PREFERRED EMBODIMENTS

Hereinafter, the present invention will be explained in more detail.

1. Preparation of Scutellaria Radix Extract

A Scutellaria Radix extract can be prepared in accordance with conventional processes. For example, the root of Scutellaria Radix can be extracted using an appropriate solvent such as water, lower alcohol, etc. In Examples of the present invention, dried roots of Scutellaria Radix were homogenized to 10˜20 mesh, and then charged into a round-bottomed flask equipped with a reflux condenser. The homogenized roots of Scutellaria Radix were extracted using hot water to prepare the Scutellaria Radix extract.

2. Regenerative Effect of Scutellaria Radix Extract During Differentiation of Nerve-Related Cells

The effect of the Scutellaria Radix extract on differentiation of nerve cells and the effect of the Scutellaria Radix extract on the regeneration of neurites were evaluated using neuronal stem cells (HiB5), neuroblastomas (SH-SY5Y) (which are differentiated nerve cells), and PC12 cells.

1) Induction of Differentiation

In order to evaluate the effect of the Scutellaria Radix extract on inducing differentiation of neuronal stem cells, neuronal stem cells (HiB5) were cultured under conditions for initiation of differentiation for 1 day. After the culture was treated with the Scutellaria Radix extract prepared above and further cultured for 2 days, neurite growth was observed. A positive control group was treated with bFGF to induce the differentiation into nerve cells.

As a result, the group treated with the Scutellaria Radix extract and the positive group all were differentiated into nerve cells. It was also observed that cell bodies were dwindled, and neurites were extended to be twice longer than their cell bodies. Therefore, it can be seen that the Scutellaria Radix extract has an excellent effect of promoting differentiation of neuronal stem cells into nerve cells.

2) Effect on Neurite Regeneration

In order to evaluate the effect of the Scutellaria Radix extract on neurite regeneration, SH-SY5Y and PC12 cells were used in accordance with the same manner as described above. Retinoic acid and NGF inducing neurite growth were used as positive control groups. It was observed that the Scutellaria Radix extract exhibits a regenerative effect on neurites in SH-SY5Y and PC12 cells and positive control groups.

3. Regenerative and Protective Effects of Scutellaria Radix Extract Against Apoptosis of Brain Nerve in MK-801 Model

In a young white rat brain administered with the Scutellaria Radix extract alone, apoptosis of nerve cells was not observed through TUNEL staining, unlike nerve cells of a young white rat brain damaged by MK-801. It was observed that the Scutellaria Radix extract considerably inhibits apoptosis of nerve cells induced by MK-801. Further, it was observed that bcl-2 mRNA, an anti-apoptosis gene, was increased in cerebral tissues by administration of the Scutellaria Radix extract.

The mechanisms by which nerve cell growth factors inhibit apoptosis of nerve cells are as follows: 1) inhibition of death effector gene expression, and 2) promotion of cell survival promoting genes (e.g., bcl-2, bcl-xL, etc) expression (Helmreich, 2001). Therefore, it is assumed that the Scutellaria Radix extract functions as a nerve growth factor, and the Scutellaria Radix extract increases the production of Bcl-2, a representative anti-apoptosis protein, thereby efficiently inhibiting apoptosis of nerve cells.

4. Effect of Scutellaria Radix Extract on Regeneration of Sciatic Nerves in the Peripheral Nervous System

Since the central nervous system and peripheral nervous system are hard to regenerate, degenerative brain diseases, and persons crippled due to industrial accidents, traffic accidents and wars, have been social problems. Therefore, special attention has been paid to studies on the regeneration of nervous systems.

Schwann cells play an important role in the generation and regeneration of the peripheral nervous system. During development of embryos, Schwann cells derived from the neural crest previously divide at the sites occupied by axons. That is, axonal growth in the peripheral nervous system depends on Schwann cells. In particular, Schwann cells produce trophic factors to regulate nerve survival and neurite growth. Axons in nerve cells secrete neuregulin to increase Schwann cell survival and to regulate the ratio between axons and Schwann cells. At this time, Schwann cells receiving no influence from axons die. At the final stage of development, Schwann cells produce myelin sheaths to insulate axons and the differentiation of Schwann cells is completed.

When peripheral nerves are injured in adults suffering from neurogenesis, they undergo Wallerian degeneration at the distal stumps toward nerve endings from the injured sites. However, the proximal stumps toward cell bodies from the injured sites start to regrow. At the distal stumps toward nerve endings from the injured sites, the degenerated axons and myclin sheaths are removed. On the other hand, at the proximal stumps toward cell bodies from the injured sites, the environment is modified to promote axonal regrowth (Kwon, Y Kim, Bhattacharyya, W. V., Cheon, K., Stiles, C. D., and Pomeroy, S. L. (1997) Activation of ErbB2 during Wallerian degeneration of sciatic nerve, J. Neurosci. 17, 8293-8299; Joung, I., Kim, H. S., Hong, J. S., Kwon, H., and Kwon, Y. K. (2000) Effective gene transfer into regenerating sciatic nerves by adenoviral vectors: potentials for gene therapy of peripheral nerve injury. Mol. Cells 10, 540-545).

Immediately after nerves are damaged, Schwann cells rapidly divide. Such Schwann cell division is believed to be due to the fact that Schwann cells fail to make contact with axons, or the division is promoted by growth factors secreted from axons. During axonal regrowth, contact of Schwann cells with axons promotes axonal differentiation and regenerates myelin sheaths. Further, Schwann cells can influence axonal regeneration from a distance. For example, though nerves are cut and separated by a gap of 1 cm, axons regenerate toward the distal stumps. Such orthodromic movement of axons is possible only when living Schwann cells exist in the distal stump.

Regeneration in the peripheral nervous system occurs in accordance with the following processes: first, Schwann cells are separated from cut axons to obtain division potential (dedifferentiation), axons of nerve cells regrow from injured sites, Schwann cells insulate the regrown axons with myclin sheaths (redifferentiation), and axons grow enough to reach muscles and form neuromuscular junctions at muscle cells.

The present inventors examined whether the Scutellaria Radix extract promotes axonal regrowth, the regeneration of myclin sheaths, and the formation of neuromuscular junctions in muscle cells, in the regeneration process of sciatic nerves through which most nerve fibers pass in the peripheral nervous system.

The present inventors observed the degree of nerve regeneration 1 week, 2 weeks and 4 weeks after intraperitoneally injecting PBS (phosphate-buffered saline) or the Scutellaria Radix extract into sciatic nerves of a rat.

As a result, it was seen that the Scutellaria Radix extract promotes axonal growth and the regeneration of myelin sheaths during peripheral nerve-regeneration.

In order to see if the Scutellaria Radix influences the regeneration of nerve endings at neuromuscular junctions, 4 weeks after operation, the present inventors separated hindlimb muscle connected to sciatic nerve. As a result, it was observed in the control group that nerve endings were stained, but did not spread to muscle fibers and thus did not form neuromuscular junctions. In the group administered with the Scutellaria Radix extract, the nerve endings spread to all muscle fibers.

Therefore, it is believed that the Scutellaria Radix extract promotes axonal growth, the regeneration of myelin sheaths and the regeneration of nerve endings to form neuromuscular junctions during regeneration of peripheral nerves.

5. Role of Nerve Growth Factors and Scutellaria Radix Extract in the Nerve Regeneration

Nerve growth factors initiate the division of neuronal stem cells, regulate the divided cells into apoptosis, induce the survival of cells orthodromically moving toward target-derived growth factors and apoptosis of cells moving in a false direction to regulate the survival of presynaptic nerve cells, and regulate new synaptic formation and remodeling. Since the Scutellaria Radix extract induces the differentiation of neuronal stem cells, inhibits apoptosis and promotes neurite differentiation, it is expected that the Scutellaria Radix extract will perform functions of nerve growth factors.

The Scutellaria Radix extract had no acute toxicity and no side effects on liver functions, through in vivo experiments using white rats. The dosage for the Scutellaria Radix extract can be varied depending upon known factors, such as age, sex, body weight, disease severity and health condition of the recipient. The daily dosage is commonly in the range of 100 to 800 mg/60 kg of body weight in two or three installments. The Scutellaria Radix extract may be mixed with an appropriate carrier or excipient, or may be diluted in an appropriate diluent. Examples of the carrier, excipient and diluent include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinyl pyrrolidone, water, methylhydroxy benzoate, propylhydroxy benzoate, talc, magnesium stearate and mineral oils. The composition according to the present invention can further comprise fillers, anti-coagulating agents, lubricants, wetting agents, flavors, emulsifying agents, preservatives, etc. For fast or sustained release of active ingredients into a mammal, the composition according to the present invention can be formulated in accordance with well-known processes.

The formulation may be in dosage form such as tablets, powders, pills, sachets, elixirs, suspensions, emulsions, solutions, syrups, aerosols, soft or hard gelatin capsules, sterile water for injection, sterilized powders, etc. The composition according to the present invention may be administered through a suitable route such as oral, transdermal, subcutaneous, intravenous or intramuscular route. In the present invention, the Scutellaria Radix extract may be formulated into pharmaceutical preparations for preventing and treating nervous system diseases, or may be added to foods or beverages. The composition according to the present invention may be used as drugs or foods to treat degenerative brain diseases such as dementia, chronic epilepsy, palsy, ischemic brain diseases, Parkinson's disease and Alzheimer's disease. Examples of foods include beverages, guns, teas, vitamin complexes, health care products, etc.

### BRIEF DESCRIPTION OF THE DRAWINGS

The above and other objects, features and other advantages of the present invention will be more clearly understood from the following detailed description taken in conjunction with the accompanying drawings in which:

FIG. 1 is confocal microscopic images showing the effect of the Scutellaria Radix extract on inducing differentiation of HiB5 nerve cells. bFGF+ represents bFGF (basic fibroblast growth factor)-treated cells, and bFGF− represents bFGF-untreated cells;

FIG. 2 is a graph showing the effect of the Scutellaria Radix extract on inducing differentiation of HiB5 nerve cells;

FIGS. 3a to 3c are magnified (×200) views of PC12 cells 14 days after administering the Scutellaria Radix extract (50 μg/ml). These views show that neurites are considerably developed in PC12 cells;

FIGS. 4a to 4c are magnified views of PC12 cells 3 days after administering NGF (Nerve Growth Factor, 50 ng/ml). These views show the development of neurites in PC12 cells;

FIG. 5 is confocal microscopic images showing the effect of the Scutellaria Radix extract on neurite regeneration in human neuroblastoma SH-SY5Y, which is a differentiated nerve cell line. Retinoic acid is a positive control group which causes the neurite differentiation of SH-SY5Y,

FIG. 6 is a bar graph showing the effect of the Scutellaria Radix extract on neurite regeneration in human neuroblastoma SH-SY5Y, which is a differentiated nerve cell line;

FIG. 7 is a bar graph showing the length of neurites after treating the Scutellaria Radix extract (50 μg/ml) in cultures of PC12 cells for 14 days [1: a control group treated with physiological saline, 2: a group treated with NGF (50 ng/ml), 3: a group treated with the Scutellaria Radix extract (50 μg/ml)];

FIG. 8a is a photograph showing the expression of NGF mRNA after treating the Scutellaria Radix extract in PC12 cells. A group treated with the Scutellaria Radix extract exhibits far higher expression of NGF than normal group. FIG. 8b shows the expression of GAPDH mRNA as a control group in the quantification of mRNA (M: 100 bp DNA marker, 1: normal group, 2: a group treated with NGF (50 ng/kg) for 14 days, 3: a group treated with the Scutellaria Radix extract (50 μg/ml) for 7 days, 4: a group treated with the Scutellaria Radix extract (50 μg/ml) for 14 days);

FIGS. 9a and 9b show normal cerebral slices of 7-day old white rats stained by the TUNEL method. FIG. 9b is a magnified view (×400) of the open square indicated in FIG. 9a;

FIGS. 10a and 10b are photographs showing apoptosis of nerve cells on cerebral slices, 1 day after intraperitoneally injecting MK-801 (0.5 mg/kg) into 7-day old white rats. FIG. 10a shows total cerebral coronal slice. Black points represent cells positive to the TUNEL method, which is an apoptosis searching method capable of staining only cells exhibiting nuclear DNA-fragmentation. FIG. 10b is a magnified view (×400) of the open square represented in FIG. 10a. This Figure shows cells having died by apoptosis;

FIGS. 11a and 11b are photographs of the cerebral slices taken 3 days after intraperitoneally injecting the Scutellaria Radix extract (20 mg/kg) alone into 4-day old white rats. These figures reveal that the Scutellaria Radix extract does not induce nerve cell death. FIG. 11b is a magnified view (×400) of the open square indicated in FIG. 11a;

FIGS. 12a and 12b are representative photographs of the cerebral slices taken after pretreating the peritoneal cavity of 4-day old white rats with the Scutellaria Radix extract (20 mg/kg) alone for 3 days, followed by intraperitoneally injecting MK-801 (0.5 mg/kg) into the rat. These figures reveal that the Scutellaria Radix extract inhibits nerve cell apoptosis induced by MK-801 (0.5 mg/kg). FIG. 12b is a magnified view (×400) of the open square indicated in FIG. 12a;

FIGS. 13a and 13b are photographs of the cerebral slices taken after intraperitoneally injecting MK-801 (0.5 mg/kg) into 7-day old white rats to induce nerve cell apoptosis, followed by intraperitoneally injecting the Scutellaria Radix extract (20 mg/kg) for 5 days. These figures reveal that the Scutellaria Radix extract inhibits nerve cell apoptosis induced by MK-801 (0.5 mg/kg). FIG. 13b is a magnified view (×400) of the open square indicated in FIG. 13a;

FIG. 14 is a graph quantitatively showing the extent to which the Scutellaria Radix extract inhibits apoptosis of nerve cells induced by MK-801 (0.5 mg/kg) in the cerebral slice of white young rat [1: a group administered with MK-801 (0.5 mg/kg) alone, 2: a group administered with the Scutellaria Radix extract (20 mg/kg) alone for 6 days, 3: a group administered with MK-801 (0.5 mg/kg) and then administered with the Scutellaria Radix extract (20 mg/kg) for 6 days, and 4: a group pretreated with the Scutellaria Radix extract (20 mg/kg) for 3 days and then administered with MK-801 (0.5 mg/kg)];

FIG. 15a is a result of RT-PCR showing the expression of bcl-2 mRNA, an anti-apoptosis gene expressed in cerebral tissues of 4-day old white rats, after intraperitoneally injecting various doses of the Scutellaria Radix extract for 1 day (lane 2, 3, 4) or 3 days (lane 5, 6, 7). This figure reveals that the expression of bcl-2 mRNA is higher than in normal group (M: 100 bp DNA ladder, 1: normal group, 2 and 5: groups administered with the Scutellaria Radix extract (50 mg/kg), 3 and 6: groups administered with the Scutellaria Radix extract (20 mg/kg), 4 and 7: groups administered with the Scutellaria Radix extract (12.5 mg/kg). FIG. 15b shows the expression of GAPDH mRNA;

FIG. 16 is photographs showing the neuroregenerative effect of the Scutellaria Radix extract during reformation process of neuromuscular junctions. In the control group, nerve endings reach only one muscle fiber, but do not spread to other fibers. In the group administered with the Scutellaria Radix extract, the nerve endings reach all muscle fibers to form neuromuscular junctions; and

FIG. 17 is confocal microscopic images showing the effect of the Scutellaria Radix extract on nerve differentiation, after implanting the Scutellaria Radix extract-treated neuronal stem cells into rat brain, and 6 weeks after the implantation, fluorescence-staining the brain tissues with nerve marker NeuN.

The present invention is illustrated in greater detail below with reference to Examples. These Examples are provided only for illustrative purposes, but are not to be construed as limiting the scope of the present invention.

### EXAMPLE 1

### General Methods

1) Nerve Cell Line Culture

When bFGF(20 ng/ml) was added to HiB5 cells derived from white rat embryonic hippocampus, cell survival increased and HiB5 cells differentiated into nerve cells to express marker molecules of nerve cells. Cell culture medium was prepared by adding a mixture of 10% FBS (fetal bovine serum), penicillin/streptomycin, glutamine and sodium pyruvate (0.11 g/L) to DMEM. On differentiating at 39 C, another cell culture medium was prepared by adding pyruvate to a serum-free medium (N2, containing DMEM/F12, insulin, transferrin, Putreseine and BSA; Botten Stein & Sato., 1979).

PC12 cells and SH-SY5Y cells were incubated in DMEM supplemented with 10% FBS. In order to differentiate the cells, NGF or retinoic acid was treated in a serum-free medium.

2) Immunohistochemistry

Tissue sections were fixed with 4% paraformaldehyde and cryosected to a thickness of 401 μm. The cryosected tissue sections were stained with nerve cell- or astrocyte-labeled antibody and FITC-labeled secondary antibody before examining under a confocal microscope. In order to stain with nerve cell-labeled antibody, cultured cells were fixed with 4% paraformaldehyde for 20 minutes, permeated in 0.5% NP-40 for 5 minutes, and blocked using 1% BSA solution for 30 minutes. After reacting with a primary antibody at a temperature 4 C for 12 hours and then further reacting with FITC-labeled secondary antibody or rhodamin (TRITC)-labeled secondary antibody for 1 hour, the cultured cells were fixed before examining under a confocal microscope.

3) Sciatic Nerve Crush in White Rat

After a Sprague-Dawley white rat (male, weighing about 200 g) was anesthetized with pentobarbital (50 mg/kg), the left sciatic nerve was exposed at the sciatic notch. Subsequently, all nerve fibers except the artery in the sciatic nerve were cut, or both sides of nerve fibers were tied using #9 blood vessel suture, and then the center of the nerve fibers were cut using iridectomy scissors. In a crush model, nerve fibers were thoroughly crushed twice using a crush clip. After nerve fibers were injured, proximal stumps and distal stumps were obtained over various time intervals (6 hours, 1 day, 3 day, 7 day, 14 day, 21 day and 28 day), respectively, before testing. For comparison, the right sciatic nerve was used as a control group.

### EXAMPLE 2

### Preparation of Scutellaria Radix Extract

(1) Preparation of Hot Water Extract

1) 5-year roots of Scutellaria Radix were purchased from Kyeong-dong herbal medicine market in Seoul.

2) The roots of Scutellaria Radix were washed with distilled water, and dried in the shade at room temperature in a drier while maintaining a temperature lower than 40° C. for 24 hours to remove impurities.

3) The dried roots were cut to an appropriate size, dried in a dessicator filled with silica gel for 24 hours, and homogenized to 10-20 mesh size.

4) 1 kg of homogenized roots were charged into a 3L round-bottomed flask equipped with a reflux condenser, and then 1L of distilled water was added thereto.

5) The mixture of roots of Scutellaria Radix and distilled water was heated at a temperature of 100° C. for 3 hours.

6) The mixture was allowed to cool to room temperature, and then filtered through a filter (100 mesh) to obtain 7.5L of the Scutellaria Radix extract having a concentration of 8 Brix.

7) The obtained extract was diluted to 1/60 of its initial concentration (0.2 Brix (solid content: 0.2%)) before testing.

(2) Preparation of Ethanol Extract

1) 5 year roots of Scutellaria Radix were purchased from Kyeong-dong herbal medicine market in Seoul.

2) The roots of Scutellaria Radix were washed with distilled water, and dried in the shade at room temperature or in a drier while maintaining a temperature lower than 40° C. for 24 hours to remove impurities.

3) The dried roots were cut to an appropriate size, dried in a dessicator filled with silica gel for 24 hours, homogenized to 10-20 mesh size.

4) 2 kg of homogenized roots were charged into a 3L round-bottomed flask equipped with a reflux condenser, and then 20L of ethanol was added thereto.

5) The mixture of roots of Scutellaria Radix and ethanol was heated at a temperature of 100° C. for 3 hours.

6) The mixture was allowed to cool to room temperature, and then filtered through a filter (100 mesh) to obtain 17L of the Scutellaria Radix extract having a concentration of 4 Brix.

7) The obtained extract was concentrated under vacuum to evaporate ethanol, and then distilled water was added thereto to obtain the Scutellaria Radix extract having a concentration 20 Brix. The obtained extract was diluted to 0.2% before testing.

### EXAMPLE 3

### Regenerative Effect of Scutellaria Radix Extract on Differentiation of Various Nerve-Related Cells

1) Induction of Differentiation

In order to identify the effect of Scutellaria Radix extract on inducing differentiation of neuronal stem cells, HiB5 cells were cultured under conditions for initiation of differentiation for 1 day. Thereafter, the culture was treated with the Scutellaria Radix extract (50 μg/ml) and further cultured for 2 days. The cultured cells were immunostained with nerve cell-specific labeled molecule, and then neurite growth was observed under a confocal microscope. A positive control was treated with bFGF (20 ng/ml) under the same condition as described above to induce the differentiation into nerve cells. The differentiation degree was measured by double-staining neurites with nerve cell-specific labeled molecule (anti-neurofilament antibody) and FITC-labeled secondary antibody (green), followed by staining cell nuclei with propidium iodide (red).

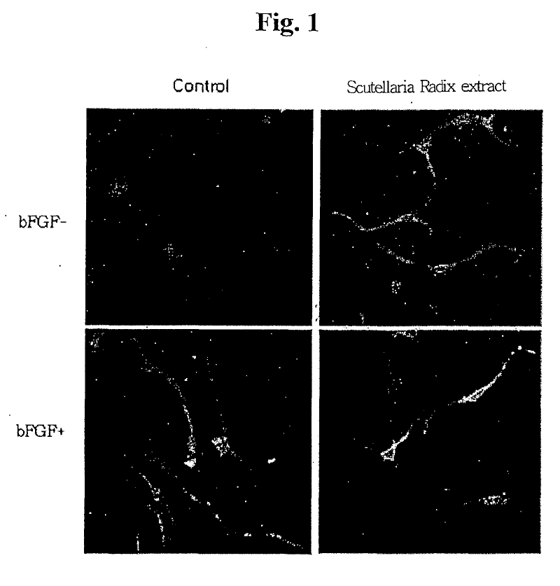

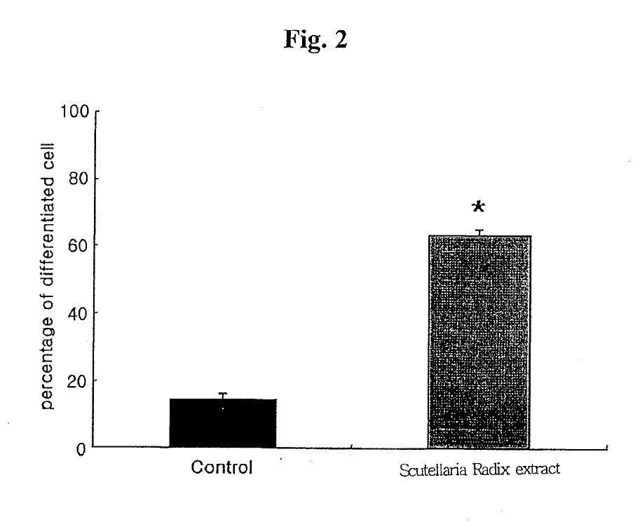

As shown in FIG. 1, treatment with bFGF induced the differentiation into nerve cells. At this time, cell bodies got smaller and neurites got longer. The group treated with the Scutellaria Radix extract showed the same changes as the group treated with bFGF, and the number of differentiated nerve cells in the treated group was about 4 times higher than in the control group (see, Table 1 and FIG. 2).

TABLE 1

| | N2 | Scutellaria Radix extract |
|---|---|---|
| Average number of | 3.07/22.38 | 13.42/21.00 |
| differentiated cells/ | | |
| total number of cells | | |
| Average (%) | 14.46 | 63.55 |

2) Effect on Neurite Regeneration

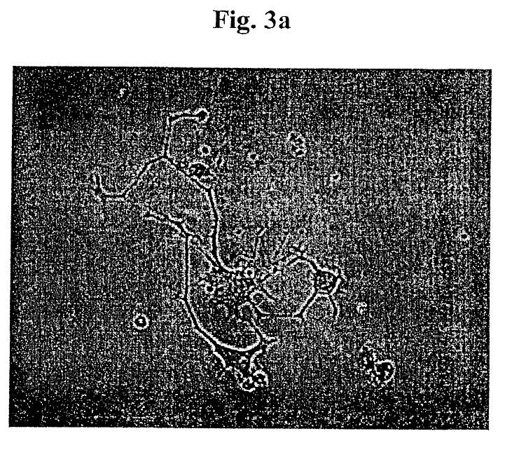

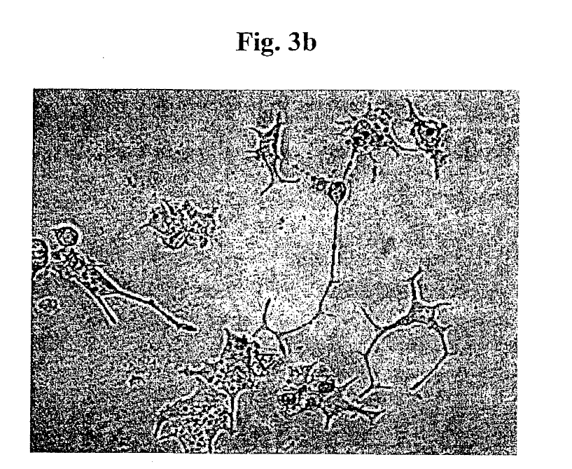

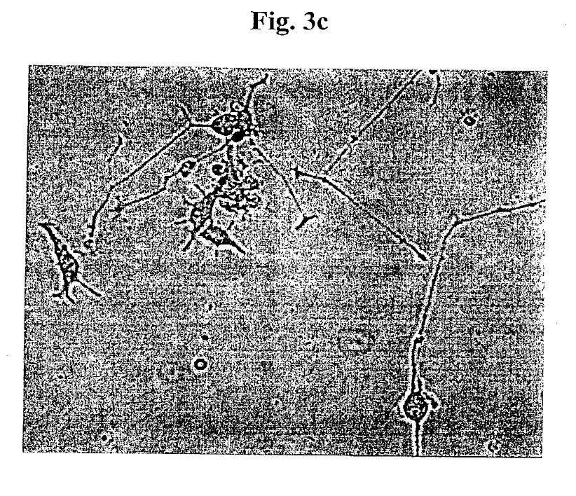

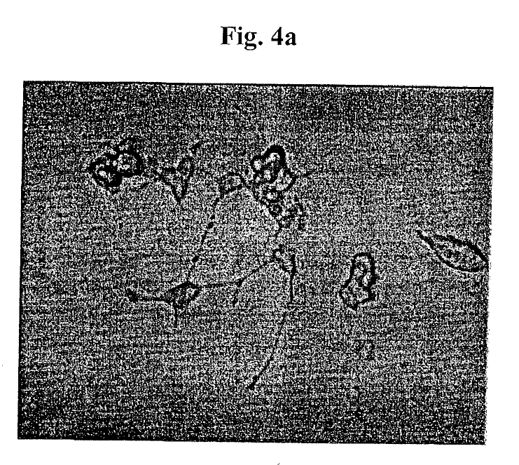

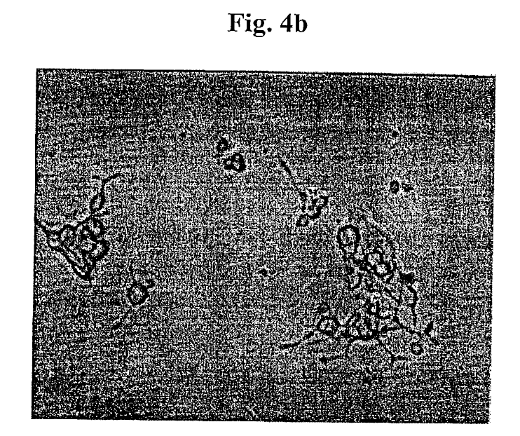

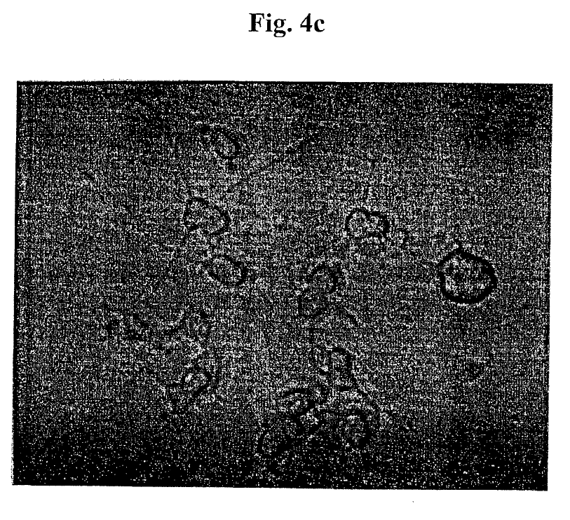

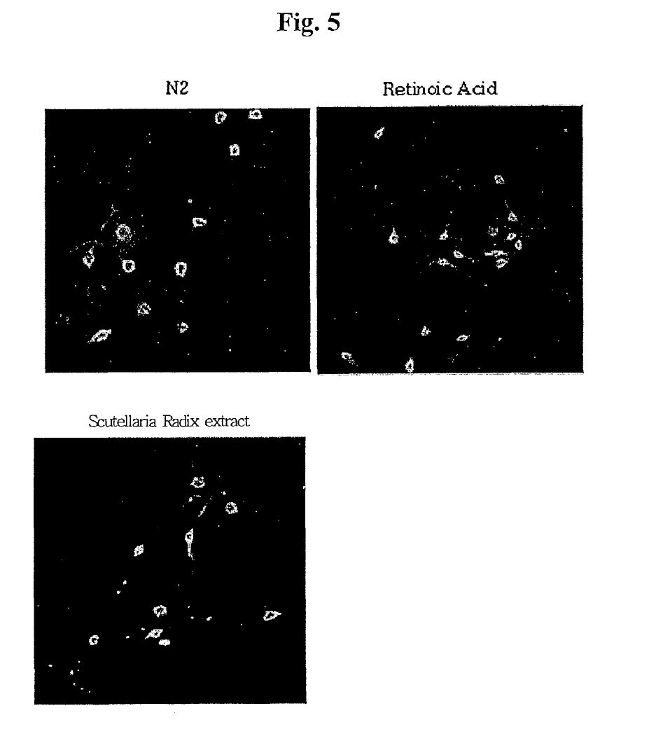

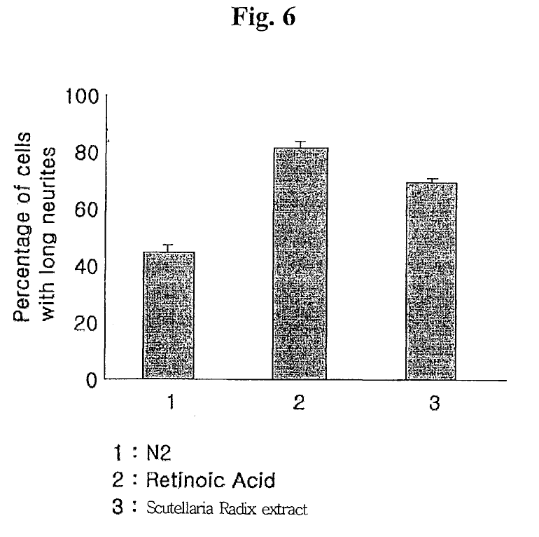

In order to examine the effect of the Scutellaria Radix extract on neurite regeneration, SH-SY5Y and PC 12 were used as differentiated nerve cell lines. Retinoic acid (50 μM) and NGF (50 ng/ml) inducing neurite growth were used as positive controls. It was observed that the Scutellaria Radix extract (each 50 μg/ml) exhibits the effect on neurite regeneration in SH-SY5Y and PC12 cells and positive control groups (see, FIGS. 3 to 5). In particular, in the case of treating with the Scutellaria Radix extract, cells having neurites three times longer than their cell bodies were about 1.5 times more than the control group in their number (see, Table 2 and FIG. 6).

TABLE 2

| | | | Scutellaria Radix |
|---|---|---|---|
| | N2 | Retinoic acid | extract |
| Average number of | 22.30/42.33 | 18.91/24.75 | 25.06/35.06 |
| differentiated cells/ | | | |
| total number of cells | | | |
| Average (%) | 44.47 | 81.59 | 69.25 |

### EXAMPLE 4

### Quantitative Comparison of Neurites in PC12 Cell Line

1) A group was treated with the Scutellaria Radix extract (50 μg/ml) alone, another group was treated with physiological saline alone, and last group was treated with NGF (50 ng/ml), and then cultured in cultures of PC12 cell line, respectively, for more than 2 weeks. Subsequently, the length of neurites in each group was measured.

2) Differentiation index was scored as follows: no neurite expression (0), the length of expressed neurites was less than the diameter of cell bodies (1), the length of expressed neurites was similar to the diameter of cell bodies (2), the length of expressed neurites was less than two times as long as the diameter of cell bodies (3), and the length of expressed neurites was more than two times as long as the diameter of cell bodies, or the expressed neurites form synapses together with other nerve cells (4). 200 differentiated cells from each microculture well were defined as one unit, and five units were statistically analyzed.

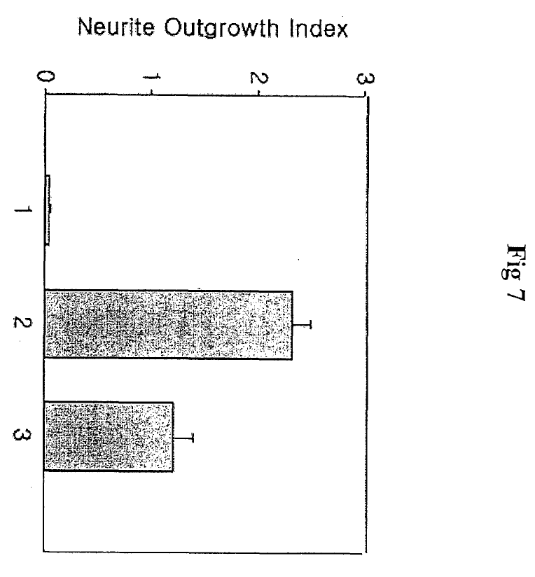

3) The results are shown in FIG. 7. As shown in FIG. 7, the group treated with the Scutellaria Radix extract shows excellent neurite formation, compared with the group treated with physiological saline.

### EXAMPLE 5

### Expression of NGF mRNA and GAPDH mRNA in PC12 Cell Line

1) A group was treated with the Scutellaria Radix extract (50 μg/ml), another group was treated with physiological saline and final group treated with NGF (50 ng/ml), and then cultured in cultures of PC12 cell line, respectively, for more than 2 weeks, and then the expression of NGF mRNA and GAPDH mRNA (a control group) was assayed by RT-PCR.

* RT-PCR

i) Total RNA Isolation

1 ml of TRI Reagent (Molecular Research Center Inc., USA) was added to 100 mg of tissue sections, and the mixture was homogenized and then left at room temperature for 10 minutes. 0.1 ml of BCP (Sigma, USA) was added to 1 ml of the homogenized mixture, mixed with each other for 1 minute, and then left at 4° C. for 10 minutes. After the mixture was centrifuged at 12,000 rpm, 4° C. for 15 minutes, the supernatant was added to cold isopropanol and left at a temperature of −20° C. for 16 hours. Thereafter, the supernatant was centrifuged at 12,000 rpm, 4° C. for 15 minutes to obtain RNA precipitates. The obtained RNA precipitates were washed with DEPC (diethylpyrocarbonate)-treated cold ethanol (75%), and dried using SpeedVac. The dried RNA was dissolved in DEPC-treated distilled water. After the concentration and purity of RNA were spectrophotometrically measured at 260 nm, the isolated RNA was stored at a temperature of −20° C. before use.

ii) cDNA Synthesis (Reverse Transcription: RT)

2 μg of total RNA obtained above was mixed with 4.0 μl of 5×RT buffer, 1.0 μl of oligo (dT16) (100 pmoles/μl), 4 μl of 10 mM dNTPs (Promega, USA), 0.5 μl of RNasin (40 Units/μl, Promega, USA) and 1.0 μl of MMLV reverse transcriptase (200 units/μl, Promega, USA), and DEPC-treated distilled water was added thereto until a total volume of the reaction solution was 30 μl. The reaction was performed in a DNA thermal cycler (Perkin Elmer 2400, USA) at 42° C. for 1 hour to synthesize cDNA.

iii) Polymerase Chain Reaction: PCR

1 μl of RT product was mixed with sense and antisense primers (each 10 pmoles), 1 μl of 10 mM dNTPs, 2 μl of 10× buffer (20 mM Tris-Cl, 1.5 mM MgCl₂, 25 mM KCl, 0.1 mg/ml gelatin, pH 8.4) and 1 unit of Taq DNA polymerase (Promege, USA), and then distilled water was added thereto until a total volume of the reaction solution was 25 μl. Polymerase chain reaction was performed using a DNA thermal cycler (Perkin Elmer 2400, USA).

iv) Electrophoresis and Analysis

10 μl μof amplified PCR product was electrophoresed in a 1.5% agarose gel, and the density was measured using a gel documentation system (Bio-Rad Lab, USA).

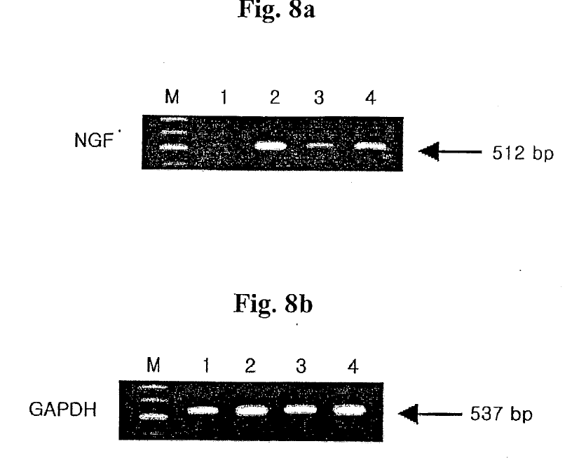

2) The results are shown in FIGS. 8a and 8b. As shown in FIGS. 8a and 8b, the group treated with the Scutellaria Radix extract shows high NGF expression, compared with the group treated with physiological saline.

### EXAMPLE 6

### Regenerative and Protective Effects of Scutellaria Radix Extract on Cranial Nerve Cells Using MK-801 Model

1) MK-801-Induced Nerve Cell Apoptosis

MK-801 reaches maximal concentrations in plasma and brain within 10 to 30 minutes of injection with an elimination half-life of 1.9 hr (Vezzani, A., Serafini, R., Stasi, M. A., Caccia, S., Conti, I., Tridico, R. V. and Samanin, R. (1989) Kinetics of MK-801 and its effect on quinolinic acid-induced seizures and neurotoxicity in rats. J Pharmacol Exp Ther 249, 278-83). Ikonomidou et al. found that when MK-801 was administered to a young rat (7˜8-days old) to inhibit NMDA receptors (for 2-3 hours), nerve cells highly sensitive to NMDA receptors died through apoptotic neurodegeneration. At this time, the number of dead nerve cells amounted to 12˜26% of total nerve cells (Ikonomidou, C., Bosch, F., Miksa, M., Bittigau, P., Vockler, J., Dikranian, K., Tenkova, T. I., Stefovska, V., Turski, L. and Olney, J. W. (1999) Blockade of NMDA receptors and apoptotic neurodegeneration in the developing brain. Science 283, 704).

2) The protective effect of the Scutellaria Radix extract on nerve cells was evaluated using models for apoptosis of nerve cells induced by MK-801 in 7-day old white rats.

Young rats were divided into 5 groups: a) a group administered with physiological saline alone, b) a group administered with MK-801 (0.5 mg/kg) alone, c) a group administered with the Scutellaria Radix extract (20 mg/kg) alone, d) a group pretreated with the Scutellaria Radix extract (20 mg/kg) and then administered with MK-801 (0.5 mg/kg), and e) a group pretreated with MK-801 (0.5 mg/kg) and then administered with the Scutellaria Radix extract (20 mg/kg). All groups were intraperitoneally injected.

Experimental animals were sacrificed under anesthetization and their brains were excised. The excised brains were fixed with formalin and tissue sections were obtained. The tissue sections were stained by TUNEL (terminal deoxynucleotidyl transferase-mediated dUTP nick-end labeling) method (Gavrieli et al, 1992), and photographed (X 1.25 and X 400) using an optical microscope (Olympus BX 50). The results are shown as follows:

a) The Group Administered with Physiological Saline Alone

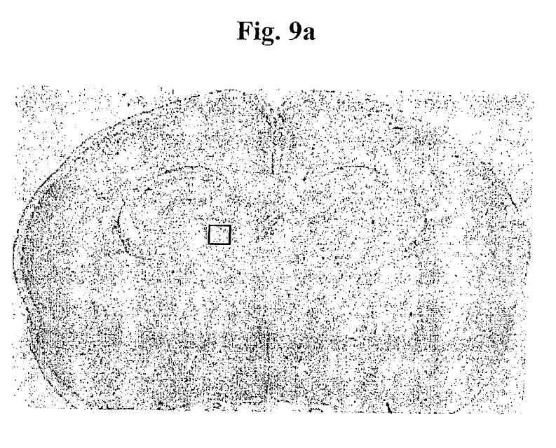

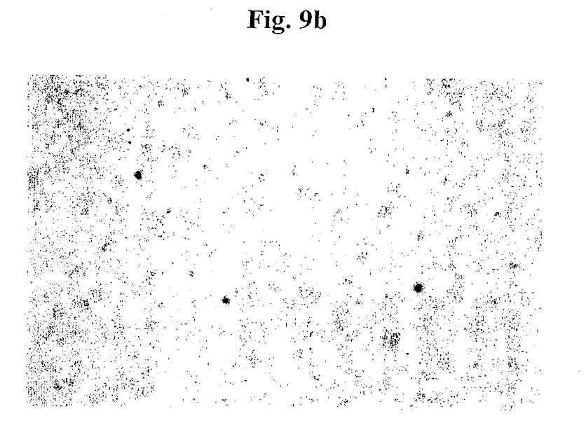

FIGS. 9a and 9b show TUNEL staining in normal cerebral sections of 7-day old white rats.

b) The Group Administered with MK-801 Alone

After intraperitoneally injecting MK-801 (0.5 mg/kg) into 7-day old white rats, apoptosis of nerve cells in cerebral slices was identified.

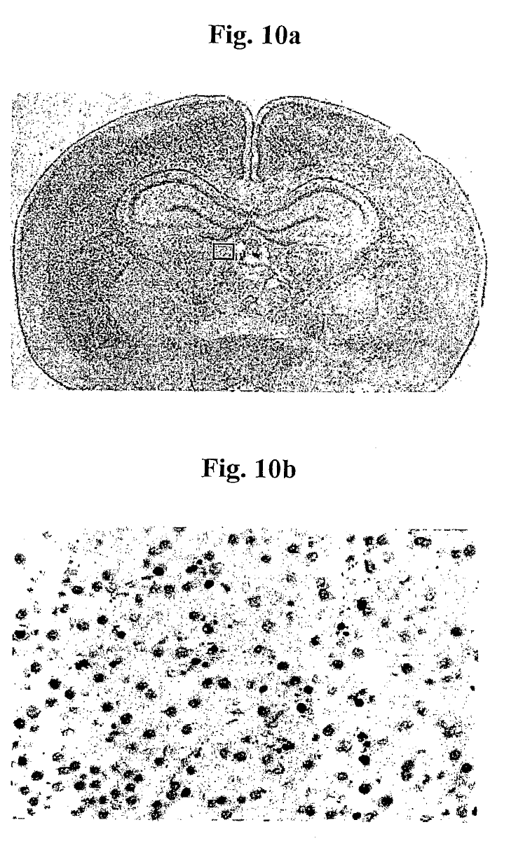

FIGS. 10a and 10b show cerebral coronal slices. Black cells represent cells positive to the TUNEL method, which stains only cells having segmented DNA in nuclei.

c) The Group Administered with the Scutellaria Radix Extract Alone

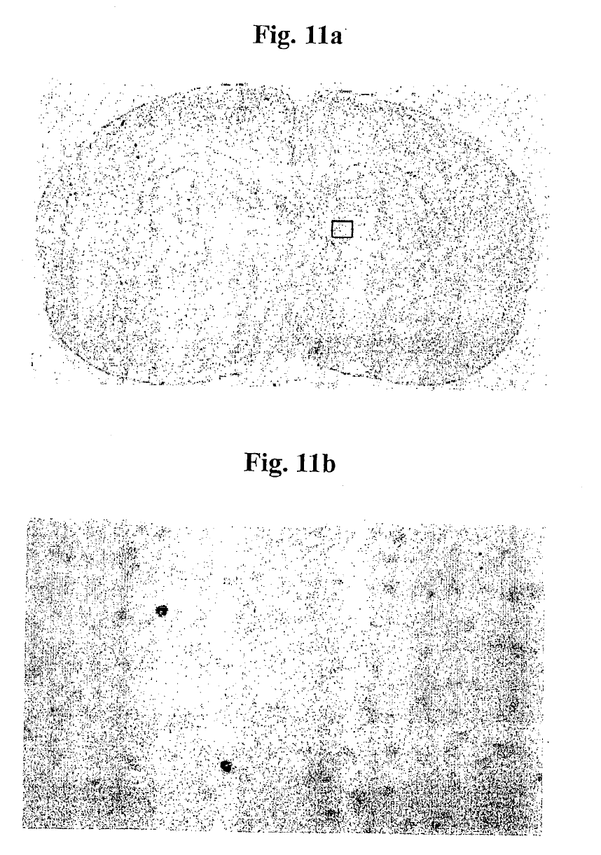

3 days after intraperitoneally injecting the Scutellaria Radix extract (20 mg/kg) into 4-day old white rats, the cerebral slices were stained with the TUNEL method. The Scutellaria Radix extract did not induce apoptosis of nerve cells (FIGS. 11a and 11b).

d) The Group Pretreated with the Scutellaria Radix Extract and then Administered with MK-801

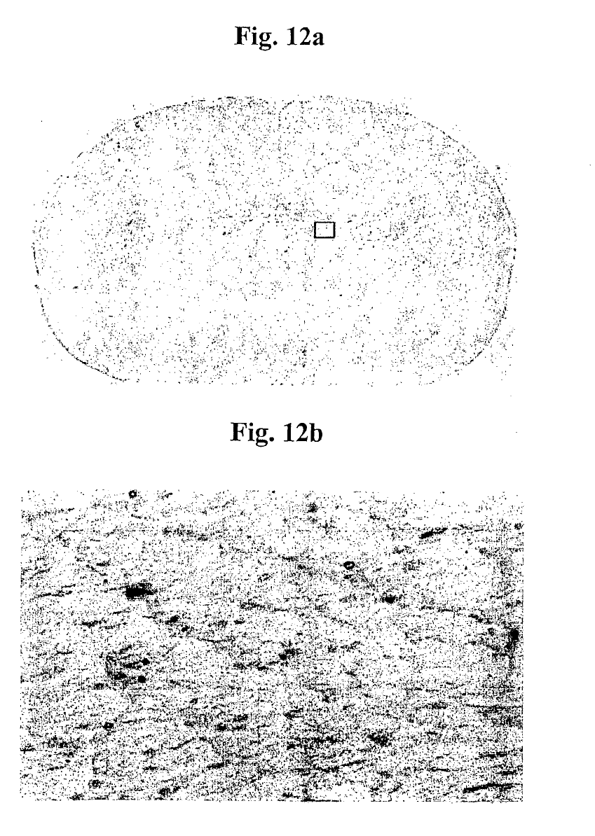

After 4-day old white rats were pretreated with the Scutellaria Radix extract (20 mg/kg) for 3 days and intraperitoneally administered with MK-801 (0.5 mg/kg) to the rat, the cerebral slices were observed. As a result, it was seen that the Scutellaria Radix extract inhibits apoptosis of nerve cells induced by MK-801 (FIGS. 12a and 12b).

e) The Group Pretreated with MK-801 (0:5 mg/kg) and then Administered with the Scutellaria Radix Extract (20 mg/kg)

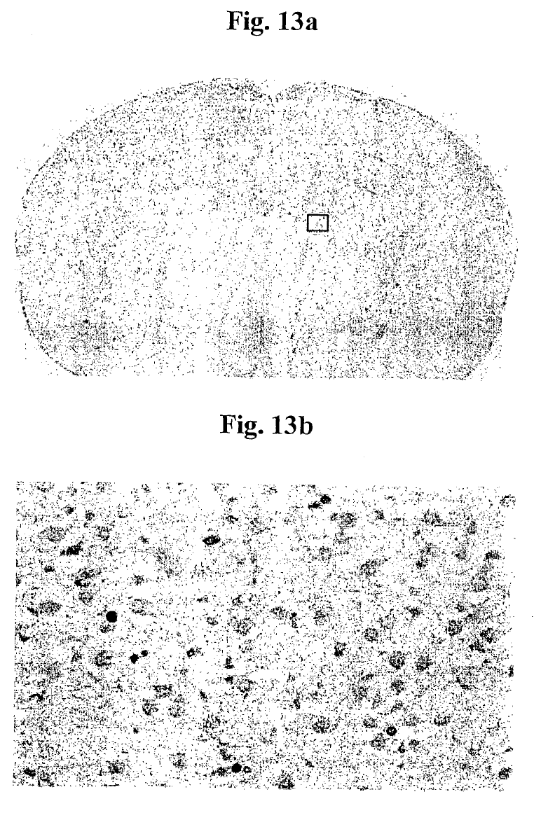

After MK-801 (0.5 mg/kg) was intraperitoneally injected into 7-day old white rats to induce apoptosis of nerve cells and intraperitoneally administered with the Scutellaria Radix extract (20 mg/kg) for 5 days, the excised cerebral slices were observed. As a result, it was seen that the Scutellaria Radix extract inhibited apoptosis of nerve cells induced by MK-801 (FIGS. 13a and 13b).

### EXAMPLE 7

### Quantitative Comparison of Nerve Cell Apoptosis in White Rat Cerebra

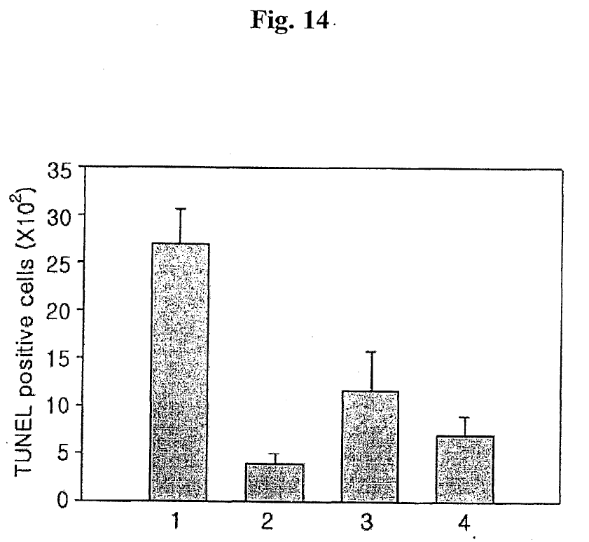

A group administered with MK-801 (0.5 mg/kg), a group administered with the Scutellaria Radix extract (20 mg/kg) for 6 days, a group pretreated with the Scutellaria Radix extract (20 mg/kg) for 3 days and then administered with MK-801 (0.5 mg/kg), and a group administered with MK-801 (0.5 mg/kg) and then administered with the Scutellaria Radix extract (20 mg/kg) for 6 days, were used to quantitatively compare the inhibition of nerve cell apoptosis by the Scutellaria Radix extract (FIG. 14). The number of TUNEL-positive dead nerve cells in the same area of cerebral slices of 12 rats per group was counted, and the numbers were averaged.

### EXAMPLE 8

### Expression of bcl-2 mRNA and GAPDH mRNA in White Rat Cerebra

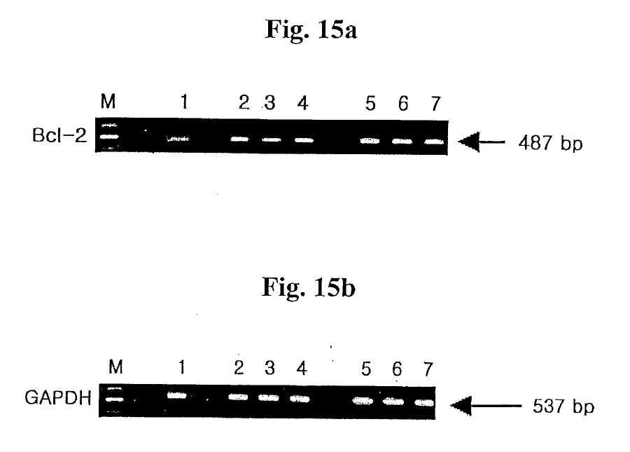

After intraperitoneally injecting 12.5 mg/kg, 20 mg/kg and 50 mg/kg, respectively, of the Scutellaria Radix extract into 4-day old white rats, RT-PCR was performed to examine the expression of bcl-2 mRNA, which is an anti-apoptosis gene expressed in cerebral tissues. As a result, in the brain tissues of rats administered with the Scutellaria Radix extract, the expression level of bcl-2 mRNA was proportional to concentration of the Scutellaria Radix extract. GAPDH mRNA was used as a control group. The expression of GAPDH mRNA was performed by RT-PCR method (FIGS. 15a and 15b).

### EXAMPLE 9

### Effect of the Scutellaria Radix Extract on Regeneration of Sciatic Nerves in the Peripheral Nervous system

After a white rat was anesthetized, its sciatic nerves were exposed and crushed. PBS or the Scutellaria Radix extract was intraperitoneally injected into the rat in an amount 2 mg per 0.1 kg of body weight. 1 week, 2 weeks and 4 weeks after suturing, nerve regeneration was observed. The rat was perfused and then sciatic nerves were obtained from the distal stump. After the obtained sciatic nerves were cryosected to a thickness of 7-10 μm, cryosected sciatic nerves were double-stained using beta-tubulin isotypeIII (cy3, red), which is an axon marker, and MBP (myelin binding protein, cy2, green) antibody, which is a differentiation (myclin) marker of Schwann cells. It was observed under a confocal microscope that axons were longer than 300 μm and myelin sheaths were longer than 200 μm.

4 weeks after operation, the number of axons longer than 300 μm had doubled. 1 week after operation, the number of myelin sheaths longer than 200 μm had doubled, and 4 weeks after operation, the number had increased by 3.5 times.

Therefore, the Scutellaria Radix extract promotes axonal growth and the regeneration of myelin sheaths during regeneration of peripheral nerves.

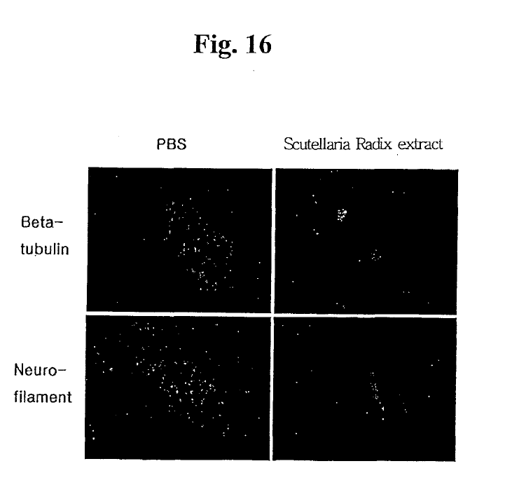

In order to see whether the Scutellaria Radix extract influences the regeneration of nerve endings in the neuromuscular junctions, 4 weeks after operation, hindlimb muscles connected to sciatic nerves were separated and cryosected. The neuromuscular junctions were stained using beta-tubulin isotypeIII and neurofilament, which are nerve markers. 4 weeks after operation, it was observed in a control group that nerve endings were stained, but did not spread to muscle fibers and thus did not form neuromuscular junctions. However, in the group administered with the Scutellaria Radix extract, the nerve endings spread to all muscle fibers (FIG. 16).

Therefore, the Scutellaria Radix extract promotes axonal growth, the regeneration of myelin sheaths and the regeneration of nerve endings to form neuromuscular junctions during regeneration of peripheral nerves.

### EXAMPLE 10

### Effect of Scutellaria Radix Extract on Differentiation of Neuronal Stem Cells Implanted into Adult Rat Hippocampus

In order to evaluate the effect of Scutellaria Radix extract on differentiation of nerve cells, hippocampus-derived neuronal stem cell line was used. HiB5 cell line used in this experiment was prepared by infecting primary cultured cells of temperature sensitive SV40 large T antigen in rat embryonic hippocampus (embryonic day 16) using retroviral vectors. The HiB5 cell line was divided at the permissive temperature (32° C.), but the cell division stopped at the non-permissive temperature (body temperature of rat: 39° C.). A small number of GABAegic neurons differentiated in the rat embryonic hippocampus (embryonic day 16), and glutamatergic pyramidal cell precursors still divided, some of which penetrated into dentate gyrus regions through dentate migration pathways in embryonic day 18 to differentiate into glutamatergic granule cells.

In order to evaluate the effect of the Scutellaria Radix extract on differentiation of neuronal stem cells, HiB5 cells were treated with 50 μg/ml of the Scutellaria Radix extract during culturing under conditions for initiation of differentiation, and then labeled with DiI.

After an adult rat was anesthetized and its head was fixed using a stereotaxic frame, HiB5 cells (6.0×104 cells/ml) treated with the Scutellaria Radix extract and then labeled with 2 μl of DiI were injected into hippocampus on the back of the rat. 6 weeks after operation, after brain slices were fluorescence-stained with NeuN marker, the differentiation of nerve cells was examined.

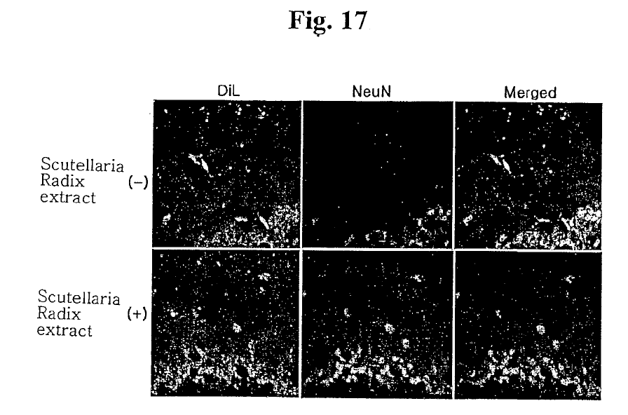

As shown in FIG. 17, DiI-labeled HiB5 cells were found around pyramidal cells on the hippocampal CA1 region, but a few HiB5 cells were differentiated into nerve cells and were stained by the NeuN marker. In the case of treating with the Scutellaria Radix extract before injecting HiB5 cells, most of DiI-labeled cells were differentiated into nerve cells and were stained by the NeuN marker. Therefore, it is believed that the Scutellaria Radix extract promotes the differentiation of neuronal stem cells, as in the cell culture experiment.

As described above, the composition according to the present invention promotes the differentiation of neuronal stem cells and the regeneration of nerve cells, thereby the nerve cells readily forming axons and dendrites. Therefore, the composition according to the present invention has excellent neuroprotective and neuroregenerative effects on nerve cells and injured nerve tissues. In addition, the composition according to the present invention can be used as a therapeutic agent for the prevention and treatment of neurodegenerative diseases or nerve injuries, in particular, dementia, Parkinson's disease, Alzheimer's disease, epilepsy, palsy, ischemic brain diseases and peripheral nerve injuries.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A composition for protecting nerve cells, promoting nerve cell growth and regenerating nerve cells, or for preventing and treating nerve injuries and nervous diseases, comprising a Scutellaria Radix extract.

2. A composition for pretreating nerve cells with a Scutellaria Radix extract in order to protect nerve cells, promote nerve cell growth and regenerate nerve cells, or to prevent and treat nerve injuries or nervous diseases.

3. The composition as set forth in claim 1 or 2, wherein the Scutellaria Radix extract is extracted from root of Scutellaria Radix using hot water.

4. The composition as set forth in claim 1 or 2, wherein the Scutellaria Radix extract is extracted from root of Scutellaria Radix using ethanol.

5. The composition as set forth in claim 1 or 2, wherein the nerve cells are neuronal stem cells.

6. The composition as set forth in claim 1 or 2, wherein the nerve injuries and nervous diseases are brain injuries and brain diseases, respectively.

7. The composition as set forth in claim 6, wherein the brain injuries or brain diseases include dementia, Parkinson's disease, Alzheimer's disease, Huntington's disease, epilepsy, palsy, stroke, ischemic brain diseases, degenerative brain diseases and memory loss.

8. The composition as set forth in claim 1 or 2, wherein the nerve injuries or nervous diseases include peripheral nerve injuries, amyotrophic lateral sclerosis and peripheral nervous diseases.

9. The composition as set forth in any one of claims 1 to 8, wherein the composition is used as foods or drugs.
